(19) United States
(12) Patent Application Publication
(10) Pub. No.: US 2008/0160043 A1
(43) Pub. Date: Jul. 3, 2008

(54) PREPARATION METHOD OF BETA-GLUCAN FROM SCHIZOPHYLLUM COMMUNE AND COMPOSITION FOR EXTERNAL APPLICATION COMPRISING THE SAME

(76) Inventors: Moo-Sung KIM, Suwon-si (KR); Yong-Dae PARK, Cheongju-si (KR); Sang-Rin LEE, Seoul (KR)

Correspondence Address: BIRCH STEWART KOLASCH & BIRCH, PO BOX 747, FALLS CHURCH, VA, 22040-0747, US

(21) Appl. No.: 11/778,489

(22) Filed: Jul. 16, 2007

(51) Int. Cl. A61K 36/06; A61P 17/00; C12P 19/04

(52) U.S. Cl. 424/195.15; 435/101

(57) ABSTRACT

Disclosed herein is a method for high-yield production of Schizophyllum commune-derived beta-glucan having a homogeneous composition, comprising subjecting mycelia of Schizophyllum commune to liquid culture with an addition of a synthetic adsorbent, and a composition for external application comprising the beta-glucan produced therefrom, which is capable of relieving dry skin conditions, atopic diseases and itching.

### BACKGROUND OF THE INVENTION

(a) Field of the Invention

The present invention relates to a method for production of beta-glucan from Schizophyllum commune and a composition for external application comprising the beta-glucan produced therefrom. More specifically, the present invention relates to a method for mass production of beta-glucan from Schizophyllum commune within a short period of time and a composition for external application comprising the beta-glucan produced therefrom, which is intended for alleviation of itching and pain caused by dry skin disorders, atopic diseases and bacterial infections.

(b) Description of the Related Art

Skin is the primary defense which functions to protect body organs against changes of weather including temperatures and humidity, and external stimuli such as UV, pollutants, and the like. However, excessive physical/chemical stimuli and stress from external environments, malnourishing and malnutrition, and the like may result in deterioration of the normal function of skin and damage to the skin.

In order to keep the skin healthy and beautiful by preventing such adverse events, a great deal of efforts has been made to effectively suppress the skin damage by maintenance of normal skin function and activation of skin cells via the use of beneficial physiologically active substances obtained from various animals, plants and microorganisms. Throughout the world, a great deal of research has recently focused on applications of functions of mushroom-derived physiologically active substances, beta-glucans, for the prevention of skin aging.

Taxonomically speaking, Schizophyllum commune is a wood-rotting fungus that belongs to Agaricales, Tricholomataceae, Schizophyllum of basidiomycetes. In the traditional herbal medicine, Schizophyllum commune has been considered to have sweetness and mildness in characteristics thereof and has been used in health improvement of people with weak constitution and in the treatment of various gynecological diseases including leucorrhea.

Schizophyllum commune produces a beta-1,6-branched-beta-1,3-glucan polysaccharide having a homogeneous composition which is extracellularly secreted by liquid culture. Such a polysaccharide is called schizophyllan which has been used as an anticancer drug in the form of an injectable preparation. In recent years, it is also used as a cosmetic material for the prevention of senescence.

Japanese Unexamined Patent Publication No. Hei 05-286843 discloses a cosmetic composition having skin whitening and moisturizing effects, which utilizes a mixture extracted and concentrated from a culture of Schizophyllum commune.

Korean Patent Application Publication No. 1999-76537 A1 (Patent No. 10-0295623) describes that β-1,6-branched-β-1,3-glucan, obtained by liquid culture of the mycelia of Schizophyllum commune, exhibits preventive effects on skin aging, skin-whitening effects, and skin damage-relieving effects. Further, Korean Patent Application Publication No. 2000-55724 A1, assigned to the same applicant, discloses a moisturizing cosmetic composition comprising beta-1,6-branched-beta-1,3-glucan prepared by liquid culture of Schizophyllum commune.

In addition, U.S. Pat. No. 6,251,877 discloses skin whitening effects and skin-wrinkle reducing effects of the isolated beta-glucan.

Korean Patent No. 10-0328877 relates to a cosmetic composition, proposing that Schizophyllum commune-derived beta-glucan has a proliferative activity on skin cells and collagen fibers, a skin-regenerating activity such as healing of sun burn, and an anti-inflammatory activity.

In addition, as a medicinal use of Schizophyllum commune, Korean Patent Application Publication No. 2000-44986 A1 describes that the beta-glucan, isolated from Schizophyllum commune, attenuates or prevents drug-induced irritation or sensitization.

Further, Korean Patent No. 10-0328877 discloses anti-inflammatory effects and anti-irritation effects of Schizophyllum commune.

As such, many attempts have been actively made to find novel uses and applications of the beta-glucan as a main active ingredient of Schizophyllum commune.

To this end, the inventors of the present invention have investigated on a novel use that is capable of ameliorating skin dryness and atopic diseases and is capable of reducing viral infection-induced itching, other than focusing on skin moisturizing, anti-aging, and anti-inflammatory action of Schizophyllum commune.

Cells present on the outer layer of skin are peeled off from the skin and new keratinocytes function in place of such defoliated cells. Such a repeated and serial changing process in the skin cells is called keratinization.

Keratinocytes during the keratinization process form a stratum corneum while producing natural moisturizing factors (NMFs) and intercellular lipids (ceramides and the like), which thereby results in solidity and flexibility of the stratum corneum that consequently will have a function as the skin barrier.

However, the stratum corneum is highly susceptible to functional loss or damage by various factors, e.g., living and behavioral factors such as excessive face washing and bathing, environmental factors such as dry atmosphere, contaminants, etc, and endogenous disorders such as atopic dermatitis and senile pruritus, formation of vesicles due to viral infections, and the like.

With recently substantial increases of various harmful factors, there is a gradual increase in the number of patients who complain of dry skin conditions and consequential disorders.

Dry and sensitive skin has low moisture-retention ability and low recovery of normal function from such a low water-holding capacity and thereby is vulnerable to or may suffer from skin keratinization and pruritus. Severe cases of such conditions may lead to development of dermatitis such as atopic dermatitis.

As a measure to treat the skin dryness and pruritus including atopic dermatitis, several compositions have been proposed. However, all of these compositions are pharmaceutical compositions, and most of them contain steroidal drugs or antibiotics as an active ingredient. Therefore, chronic application of the drug composition to skin may result in a high risk of serious side effects such as occurrence of telangiectasia and/or thickening or extension of the stratum corneum.

The pharmaceutical composition of Korean Patent Application Publication No. 2000-46633 A1 comprising, as a main active ingredient, a gamma-linolenic acid which is recently widely used in the alleviation of atopic dermatitis, suffered from problems such as poor stability due to easy oxidation, and a difficulty in application thereof to sensitive skin, due to relatively high irritability to the skin.

Meanwhile, despite frequent incidence of dermal or mucosal damage caused by viral infections such as varicella-zoster virus (VZV), herpes simplex virus (HSV) and Enterovirus, there is not yet established an effective therapeutic method for the treatment of such conditions. In order to prevent secondary infections, the affected part is treated with administration of an antibiotic or is sterilized with povidone iodine and the like.

However, to the best of our knowledge, there is no case of an external preparation that is capable of soothing pain and itching of the affected parts while ameliorating viral infection-induced damage including vesicles, lesions and ulcers by viral infections such as infections with varicella-zoster virus (VZV), herpes simplex virus (HSV) or Enterovirus.

In recent years, various anti-viral drugs were developed, but it cannot be said that these anti-viral preparations are sufficient for amelioration of viral infection-induced damage including vesicles, lesions and ulcers, particularly suppression of pain and itching at the affected parts.

As a result of a variety of extensive and intensive studies and experiments to solve the problems as described above, the inventors of the present invention have confirmed that Schizophyllum commune-derived beta-glucan is effective for mitigation of dry skin conditions, amelioration of atopic conditions, and relief of virus-induced itching including chicken pox.

On the other hand, a variety of research and study has been actively undertaken for the development of novel uses and production methods on Schizophyllum commune which is utilized for various applications including the aforementioned cosmetic materials, pharmaceutical compositions and the like.

Generally, production of beta-glucan (schizophyllan), which is a main active ingredient of Schizophyllum commune, is carried out by a method involving culturing the mycelia of Schizophyllum commune in a suitable medium and then recovering the resulting beta-glucan. Fungi and mushrooms including Schizophyllum commune exhibit unique growth characteristics in which the substrate (vegetative) hyphae adhere to the substrate and aerial hyphae spread and grow away from the substrate hyphae. Therefore, many of fungi grow in the form of aggregates or pellets in a liquid medium and exhibit a tendency to interfere with the uniform production of desired products. So, in order to overcome such problems, various attempts have been made which include enhancement of the culture conditions such as agitation speed or an addition of surfactants, but such trials were not practically applied.

Upon production of beta-glucan from Schizophyllum commune, Schizophyllum commune grows as the mycelia in the form of a spherical pellet or of a pellet aggregate, while producing beta-glucan. Such a form of the mycelia exhibits a difference in a degree of aging between internal and external constituent hyphae and a difference in absorption and availability of medium components, which thereby result in a reduction of the overall product productivity.

Further, culturing of Schizophyllum commune is also accompanied by production of various by-products, for example, glucan-degrading enzyme, i.e. endo-1,3-beta-glucanase, and (4E,8E)-N-D-2′-hydroxypalmitoyl-1-O-beta-D-glucopyranosyl-9-methyl-4,8-sphingadienine and (4E,8E)-N-D-2′-hydroxystearoyl-1-O-beta-D-glucopyranosyl-9-methyl-4,8-sphingadienine (Mizushina et al. 1998. Biochem. Biophys. Res. Commun. vol. 249, pp. 17), which are cerebrosides known as FIS (fruiting-inducing substance). Endoglucanase (Prokop et al. 1994. Can. J. Microbiol Rev. vol. 40, no. 1, pp. 18) is produced at a late stage of the Schizophyllum commune culturing process, and degrades the already produced β-1,6-branched-β-1,3-glucan, thereby decreasing a production yield of a desired product. In addition, the above-mentioned FIS is a substance that suppresses the mycelial growth and induces the formation of a fruiting body, and interruption of the mycelial growth by FIS also leads to no production of beta-glucan (Wessels. 1978. Genetics and Morphogenesis in the Basidiomycetes. pp. 81-104. Academic Press; and Prokop et al., 1992. Experimental Mycology. vol 16. pp. 197-206).

To this end, the present inventors have made an attempt to develop a method for more efficient production of beta-glucan as a main active ingredient of Schizophyllum commune. For this purpose, we achieved improved productivity of products by taking advantage of dual effects, i.e. the adherence as a physiological property of the mushroom mycelia and the removal of by-products during a culture process, rather than focusing on simple development of a medium composition.

### SUMMARY OF THE INVENTION

Therefore, the present invention has been made in view of the above problems, and it is an object of the present invention to provide a composition for external application which is capable of alleviating skin dryness and atopic diseases and reducing pruritus caused by bacterial infections.

It is another object of the present invention to provide a method for high-yield production of beta-glucan from Schizophyllum commune.

### DESCRIPTION OF DRAWINGS

The above and other objects, features and other advantages of the present invention will be more clearly understood from the following detailed description taken in conjunction with the accompanying drawings, in which:

FIG. 1 is a graph showing changes in mycelial mass, polysaccharide production and glucose consumption over time, as carried out in Example 1;

FIG. 2 is a graph showing changes in mycelial mass, polysaccharide production and glucose consumption over time, as carried out in Comparative Example 1;

FIG. 3 is a graph showing changes in mycelial mass, polysaccharide production and glucose consumption over time, as carried out in Comparative Example 3;

FIG. 4 is a graph showing an expression amount of filaggrin by Western blotting analysis;

FIG. 5 is a graph showing a recovery of moisture loss over time by beta-glucan; and

FIG. 6 is a graph showing percent survival of mice after infection of animals with Staphylococcus aureus.

### DETAILED DESCRIPTION OF THE INVENTION

In accordance with an aspect of the present invention, the above and other objects can be accomplished by the provision of a composition for external application having relieving effects on skin dryness conditions, pruritus and pain, and comprising Schizophyllum commune-derived beta-glucan, wherein the content of the Schizophyllum commune-derived beta-glucan is in a range of 0.01 to 20% by weight, based on the total weight of the composition.

The beta-glucan is a homogeneous beta-1,6-branched-beta-1,3-glucan, and has a beta-1,6-residue for every three beta-1,3 main chains.

The pruritus and pain are those manifested by various symptoms and conditions.

As an example, the pruritus and pain may be atopic pruritus and pain, or those caused by a secondary bacterial infection. The secondary bacterial infection is an infection by any one selected from the group consisting of Staphylococcus aureus, Streptococcus pyogenes, and any combination thereof Further, the pruritus and pain may be caused by bacterial infection, specifically Staphylococcus aureus.

Further, the pruritus and pain may be caused by viral infection. The viral infection may be caused by chicken pox virus, herpes zoster virus, or herpes simplex virus.

Further, the pruritus and pain may include senile pruritus, and pruritus and pain in vesicles, lesions or ulcers of skin mucosa by bacterial or viral infections.

In accordance with another aspect of the present invention, there is provided a method for producing Schizophyllum commune-derived beta-glucan, comprising subjecting mycelia of Schizophyllum commune to liquid culture with an addition of a synthetic adsorbent.

Specifically, the Schizophyllum commune-derived beta-glucan is produced by a process comprising:

1) inoculating a seed obtained by culturing the mycelia of Schizophyllum commune in an amount of 1 to 10% (v/v) into a liquid medium;

2) culturing the mycelial cells for 4 to 8 days with an addition of a sterile synthetic adsorbent in an amount of 1 to 10%, based on the total weight of the medium;

3) centrifuging the thus-obtained culture solution to remove the mycelia and the synthetic adsorbent; and

4) recovering, separating and purifying beta-glucan from the resulting beta-glucan solution.

The liquid medium may be adjusted to contain 1 to 20% glucose, 0.1 to 5% yeast extract, 0.1 to 5% malt extract, 0.1 to 1% ammonium sulfate ((NH₄)₂SO₄), 0.1 to 1% potassium dihydrogen phosphate (KH₂PO₄), and 0.01 to 0.5% magnesium sulfate (MgSO₄-7H₂O).

Preferably, the culturing process is carried out at a temperature of 20 to 35° C., an agitation rate of 100 to 400 rpm, and an aeration rate of 0.5 to 2 vvm.

Hereinafter, the present invention will be described in more detail.

Upon production of beta-glucan via liquid culture of the mycelia of Schizophyllum commune in accordance with the present invention, an addition of the synthetic adsorbent during the cell culture process achieves further enhanced productivity via adsorption of culture by-products by the synthetic adsorbent, in conjunction with uniform cell growth and improved productivity of beta-glucan by adhesiveness of the mycelia. The thus-produced beta-glucan not only has a conventional anti-aging activity of skin, but also has various skin-protective functions, that is, being effective for alleviation of skin dryness conditions, atopic diseases and pruritus.

Depending upon the origin and production methods, mushroom beta-glucan exhibits significant differences in uniformity of a sugar composition, a degree of branching, a molecular weight, a tertiary structure and the like, which may frequently result in differences in physico-chemical properties and in vivo functions therebetween.

For example, even though beta-glucan of Ganoderma licidum is extracted largely from cell walls, beta-glucan is also extracellularly secreted in a small amount and has a bond of mannose, galactose and the like, in addition to glucose. Beta-glucan of Coriolus versicolor is extracted from cell walls, and also usually includes beta-1,4-bonding types, in addition to beta-1,3-bonds. Further, beta-glucan of Lentinus edodes is extracted from cell walls, and is beta-1,6-branched-beta-1,3-glucan having two beta-1,5 residues for every 5 beta-1,3 main chains. Meanwhile, beta-glucan of Pleurotus ostreatus is extracted from cell walls, is composed of various types of glucans, and has a bond of mannose, galactose and the like, in addition to glucose. Further, beta-glucan of Phellinus linteus is extracted from cell walls, is composed of various types of glucans, contains 70 to 90% glucose in conjunction with mannose, galactose and the like. Further, beta-glucan of Saccharomyces cerevisiae is extracted from cell walls, is substantially free of branching, and is heterogeneous and insoluble.

Schizophyllum commune-derived beta-glucan in accordance with the present invention is a homogeneous beta-1,6-branched-beta-1,3-glucan, and has a beta-1,6-residue for every three beta-1,3 main chains. As a result, the Schizophyllum commune-derived beta-glucan has a branched, homogeneous and unique structure, exhibits extracellular secretion of stable neutral polysaccharide and is composed only of glucose, whereas the beta-glucans from the above-mentioned common mushrooms have heterogeneous sugar compositions and structures.

Conventionally known major functions of Schizophyllum commune-derived beta-glucan include proliferative action on skin cells and collagen fibers, skin-regenerating action such as healing of sunburn, and anti-inflammatory action. Further, such beta-glucan was largely used as a cosmetic material.

The present inventors have made an attempt to discover and elucidate the fact that the beta-glucan prepared by the above-mentioned method has not only a conventionally-known anti-aging activity of skin, but also exhibits various skin-protective functions, that is, being effective for the alleviation of skin dryness conditions, atopic diseases and pruritus.

The composition for external application in accordance with the present invention comprises 0.01 to 20% by weight of Schizophyllum commune-derived beta-glucan, based on the total weight of the composition, and has relieving effects on skin dryness conditions, pruritus and pain.

Examples of the pruritus and pain may include atopic pruritus and pain, the atopic pruritus and pain resulting from a secondary bacterial infection, the pruritus and pain caused by bacterial infection, the pruritus and pain caused by chicken pox virus, herpes zoster virus or herpes simplex virus, senile pruritus, and the pruritus and pain in vesicles, lesions or ulcers of skin mucosa due to bacterial or viral infections. Such itching diseases can be effectively mitigated by the use of the external preparation comprising beta-glucan in accordance with the present invention.

According to Experimental Example 4 of the present invention, it was confirmed that application of the Schizophyllum commune-derived beta-glucan to human skin cells leads to an increase in an amount of filaggrin which is a precursor of a natural moisturizing factor.

Referring to Experimental Example 5, it can be seen that upon application of the Schizophyllum commune-derived beta-glucan to skin with a damaged barrier function, recovery ability from skin moisture loss is significantly increased.

With reference to Experimental Example 6 of the present invention, it can be seen that upon testing effects of beta-glucan on alleviation of itching for atopic patients, the composition using application of the beta-glucan to skin and recovery ability from skin moisture loss exhibits excellent effects on the alleviation of itching.

In addition, referring to Experimental Example 7, it was confirmed that pruritus and pain are significantly reduced upon treatment of the Schizophyllum commune-derived beta-glucan on the patients showing attendant pruritus and pain due to viral infections.

Referring to Experimental Example 9 of the present invention, it was confirmed that application of the Schizophyllum commune-derived beta-glucan leads to a significant relief of pruritus and pain of atopic patients with bacterial infections.

A weight-average molecular weight (M.W.) of such beta-glucan varies depending upon bacterial species and production methods. The beta-glucan in accordance with the present invention has a very high M.W. of 3,000,000 to 4,000,000 and may have a different tertiary structure, which provides physical and functional properties different from those of other mushrooms or beta-glucans obtained by cultivation under different culture conditions.

According to Experimental Example 1 of the present invention, MWs of Schizophyllum commune-derived beta-glucans prepared in Examples 1 and 2 were 3,500,000 and 3,000,000, respectively, which are relatively high as compared to 2,500,000 of beta-glucan prepared by a conventional liquid culture method.

As such, Schizophyllum commune-derived beta-glucan in accordance with the present invention has alleviation effects on skin dryness and itching and is thus applicable to various compositions for external application.

There is no particular limitation to formulations of the aforesaid composition. As examples of the formulations of the aforesaid composition, mention may be made of cosmetic compositions having formulations of emollient lotions (skin lotions), nourishing lotion (milk lotions), massage creams, packs or gels, and compositions for external application having formulations of body lotions, ointments, gels, creams, patches and sprays. Alternatively, the aforesaid composition may be formulated into cleaners for household use, such as shampoos, body shampoos, kitchen detergents, residential detergents, detergents for laundry, and the like.

Herein, in the external application composition of each formulation, other components with the exception of Schizophyllum commune-derived beta-glucan may be appropriately selected and mixed by those skilled in the art, depending upon formulations for external use and desired applications.

In accordance with a further aspect of the present invention, there is provided a method for producing Schizophyllum commune-derived beta-glucan, comprising culturing mycelia of Schizophyllum commune in a liquid culture medium with an addition of a synthetic adsorbent.

Production of Schizophyllum commune-derived beta-glucan in accordance with the present invention can obtain beta-glucan with a high yield from Schizophyllum commune.

Specifically, the Schizophyllum commune-derived beta-glucan is produced by a process comprising:

1) inoculating a seed obtained by culturing the mycelia of Schizophyllum commune in an amount of 1 to 10% (v/v) into a liquid medium;

2) culturing the mycelial cells for 4 to 8 days with an addition of a sterile synthetic adsorbent in an amount of 1 to 10%, based on the total weight of the medium;

3) centrifuging the thus-obtained culture solution to remove the mycelia and the synthetic adsorbent; and

4) recovering, separating and purifying beta-glucan from the resulting beta-glucan solution.

Hereinafter, each step will be specifically described in more detail.

In Step 1, the mycelia of Schizophyllum commune grown on a slant medium are aseptically homogenized and are then inoculated in a concentration of 5 to 20% (v/v) and more preferably 10% (v/v) into a liquid culture medium.

In terms of the mycelial growth, it is preferred to use a liquid nutrient medium (pH 6.0) containing 1 to 20%, preferably 5% glucose, 0.1 to 5%, preferably 1% yeast extract, 0.1 to 5%, preferably 2% malt extract, 0.1 to 1%, preferably 0.2% ammonium sulfate ((NH₄)₂SO₄), 0.1 to 1%, preferably 0.2% potassium dihydrogen phosphate (KH₂PO₄), and 0.01 to 0.5%, preferably 0.1% magnesium sulfate (MgSO₄.7H₂O).

In Step 2, the cells are cultured for 4 to 8 days by adding a sterile synthetic adsorbent in an amount of 1 to 10%, based on the total weight of the medium.

The synthetic adsorbent added during the culturing process is porous and serves as a supporting material necessary for the mycelial growth to thereby help growth of the mycelia. Further, without significant effects on components of a medium, the synthetic adsorbent adsorbs primarily endoglucanase and FIS, e.g. cerebrosides to thereby facilitate the mycelial growth and prevent the decomposition of the resulting beta-glucan, and therefore it is possible to produce beta-glucan with a higher yield.

In other words, Schizophyllum commune, one of wood-rotting fungi, adheres and grows on dry wood, and tends to show agglomeration of pellets per se even under liquid culture. The mycelia in the thus-agglomerated form exhibits a difference in a degree of aging between internal and external constituent hyphae and a difference in absorption and utilization of the medium components, which consequently result in reduction of the overall productivity. Therefore, the use of the synthetic adsorbent as in the present invention, upon culturing of Schizophyllum commune mycelia, results in better growth of the mycelia because the cells grow taking advantage of the synthetic adsorbent as a supporting material, instead of undergoing self-aggregation of pellets.

Further, endoglucanase, a by-product produced upon culturing the mycelia of Schizophyllum commune, is produced at a late stage of the Schizophyllum commune culturing process, and degrades the already produced beta-1,6-branched-beta-1,3-glucan, thereby decreasing a production yield of a target product. In addition, FIS (fruiting-inducing substance) is a substance that suppresses the mycelial growth and induces the formation of a fruiting body, and interruption of the mycelial growth by FIS also leads to no production of beta-glucan, thereby significantly reducing the productivity of the Schizophyllum commune mycelia. Therefore, the use of the synthetic adsorbent upon culturing of Schizophyllum commune mycelia, as in the present invention, can effectively remove the above-mentioned by-product endoglucanase to thereby enhance the productivity of the mycelia.

According to Experimental Examples 1 and 2 of the present invention, it was confirmed that a liquid culture method with the addition of the synthetic adsorbent leads to an about 51% increase in the production yield of a beta-glucan polysaccharide, as compared to a simple liquid culture method. Further, it was confirmed that more than 99% of the thus-obtained polysaccharide are composed of beta-glucan.

Preferably, the synthetic adsorbent in accordance with the present invention is porous, has a large adsorption surface area, and is of a spherical shape with a diameter of 0.1 to 1 mm and preferably 0.3 to 0.6 mm. If the particle diameter is higher or lower than the above-specified range, the workability decreases and it is impossible to effectively adsorb only mycelial growth inhibitors such as endoglucanase, FIS and the like. Therefore, it is preferred to use the synthetic adsorbent having the above-specified range of the particle diameter. There is no particular limitation to the synthetic adsorbent, as long as it is a synthetic adsorbent known in the art. Preferably, the synthetic adsorbent may be one selected from the group consisting of a non-ionic styrene-divinyl benzene polymer, a phenol-formalin resin, an acrylate resin, a methacrylate resin and any combination thereof.

The synthetic adsorbent may be home-made or is commercially available. Typical examples of the commercially available synthetic adsorbent may include, but are not limited to, Amberlite XAD-1, Amberlite XAD-2, Amberlite XAD-4, Amberlite XAD-7, Amberlite XAD-8, Amberlite XAD-11 and Amberlite XAD-12 (Trademark, manufactured by Rohm and Haas, USA); Diaion HP-10, Diaion HP-20, Diaion HP-30, Diaion HP-40 and Diaion HP-50 (Trademark, manufactured by Mitsubishi Chemical Industries, Ltd., Japan); and Imac syn-42, Imac syn-44 and Imac syn-46 (Trademark, manufactured by Imacti Co., Netherlands).

For higher productivity of beta-glucan, the synthetic adsorbent is added in an amount of 0.1 to 5% by weight, preferably 2% by weight, relative to the liquid culture medium. If a content of the synthetic adsorbent is lower than the above-specified lower limit, it is difficult to sufficiently achieve a desired yield. On the other hand, if a content of the synthetic adsorbent is higher than the above-specified higher limit, this is not economical without any further increase in additional effects. Therefore, the synthetic adsorbent is preferably used in the above-specified range.

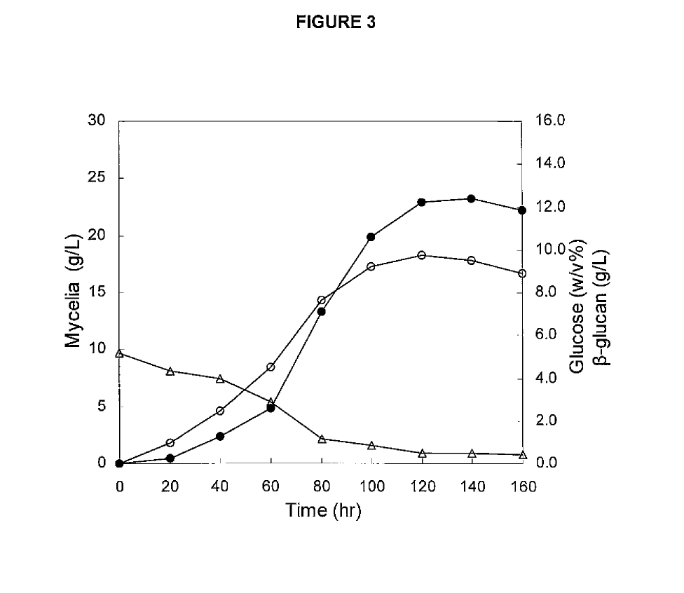

In this connection, the inventors of the present invention have proposed a method of using an activated charcoal powder upon culturing of Schizophyllum commune, in Korean Patent No. 10-0295623. In this patent, the activated charcoal is used in the form of an amorphous powder. Such activated charcoal does not selectively adsorb the mycelial growth inhibitors such as endoglucanase, FIS and the like endoglucanase, but randomly adsorbs multiple components. Further, due to no phenomenon of the attached growth as exhibited by provision of the synthetic adsorbent particles, which is proposed in the present invention, it can be seen that improvements in the productivity of beta-glucan via the use of such activated charcoal are less as compared to the synthetic adsorbent-addition culture (see FIG. 3).

After introduction of the synthetic adsorbent into the liquid culture medium, cultivation of Schizophyllum commune is carried out in a fermenter at a temperature of 20 to 35° C., preferably 28° C., an agitation rate of 100 to 400 rpm, preferably 300 rpm, and an aeration rate of 0.5 to 2 vvm, preferably 1.5 vvm, for 4 to 8 days, preferably 5 days.

In Step 3, the thus-obtained culture solution is centrifuged to remove the mycelia and the synthetic adsorbent.

The centrifugation is carried out using a conventional centrifuge, and the centrifugation condition is not particularly limited in the present invention.

In Step 4, beta-glucan is recovered, separated and purified from the resulting beta-glucan solution.

The beta-glucan solution is filtered through a filtration membrane.

Thereafter, beta-glucan is recovered by a conventional separation/purification process involving adding an ethyl alcohol to the filtrate two or three times to thereby precipitate, recover and dry the beta-glucan.

The thus-obtained Schizophyllum commune-derived beta-glucan has a relatively high molecular weight and is obtained at a higher yield, as compared to beta-glucan produced by a conventional liquid culture.

### PREFERRED EMBODIMENTS OF THE INVENTION

Now, the present invention will be described in more detail with reference to the following examples. These examples are provided only for illustrating the present invention and should not be construed as limiting the scope and spirit of the present invention.

### EXAMPLE 1

The mycelia of Schizophyllum commune grown on a slant medium were aseptically homogenized and were then inoculated in a concentration of 5% (v/v) onto a liquid culture medium (pH 6.0) containing 5% glucose, 0.5% yeast extract, 0.5% malt extract, 0.1% ammonium sulfate ((NH₄)₂SO₄), 0.1% potassium dihydrogen phosphate (KH₂PO₄), and 0.05% magnesium sulfate (MgSO₄.7FH₂O). Thereafter, sterile Amberlite XAD-7 was separately added in a concentration of 2% (v/v) to the medium which was then incubated in a 5L fermenter at a temperature of 28° C., an agitation rate of 300 rpm, and an aeration rate of 1.5 vvm for 5 days.

After the culturing was complete, the resulting culture solution was centrifuged to remove the mycelia and the synthetic adsorbent, and filtered through a press filter to obtain a solution which was free of the mycelia and the synthetic adsorbent.

The thus-obtained solution was sequentially passed through filtration membranes having a micropore diameter of 1 μm and 0.45 μm to thereby obtain a colorless or pale yellow clear culture solution from which the mycelia and the synthetic adsorbent were completely removed. Thereafter, a 4-fold volume of ethyl alcohol was added relative to the culture solution which was then left overnight at room temperature, thereby recovering the precipitates. Next, the precipitates were dissolved in the same amount of water as the initial fluid volume. In the same manner, a 3-fold volume of ethyl alcohol was gradually added to the resulting solution which was then left overnight at room temperature, thereby recovering the precipitates.

The thus-recovered precipitates were collected, subjected to hot-air drying at 80° C. to evaporate alcohol components, was dried at −70° C. using a freeze-dryer, and processed into powder using a grinder, thereby obtaining powdered beta-glucan.

### EXAMPLE 2

According to the same manner as in Example 1, beta-glucan was obtained from Schizophyllum commune. For this purpose, the mycelia of Schizophyllum commune were inoculated in a concentration of 5% (v/v) onto a liquid culture medium (pH 6.0) containing 5% glucose, 0.5% yeast extract, 0.5% malt extract, 0.1% ammonium sulfate ((NH₄)₂SO₄), 0.1% potassium dihydrogen phosphate (KH₂PO₄), and 0.05% magnesium sulfate (MgSO₄.7H₂0). Thereafter, sterile Diaion HP-20 was separately added in a concentration of 2% (v/v) to the medium which was then incubated in a 5 L fermenter at a temperature of 28° C., an agitation rate of 250 rpm, and an aeration rate of 1.25 vvm for 5 days.

### COMPARATIVE EXAMPLE 1

Beta-glucan was obtained from Schizophyllum commune in the same manner as in Example 1, except that a synthetic adsorbent was not used.

### COMPARATIVE EXAMPLE 2

Beta-glucan was obtained from Schizophyllum commune in the same manner as in Example 2, except that a synthetic adsorbent was not used.

### COMPARATIVE EXAMPLE 3

Beta-glucan was obtained from Schizophyllum commune in the same manner as in Example 1, except that activated charcoal was used, instead of a synthetic adsorbent.

### EXPERIMENTAL EXAMPLE 1

Assay of beta glucan which is contained in samples prepared in Example 1 and Comparative Example 1 was carried out with a modification of “Mushroom and Yeast Beta-Glucan Assay” (Megazyme, Wicklow, Ireland).

10 mg of an assay sample was introduced into a glass cap tube (14×120 mm) to which 3 mL of distilled water at 70° C. was then added, and the mixture was stirred. After the sample was cooled, 6 mL of 99% ethyl alcohol was added. Upon formation of precipitates, centrifugation was carried out at 2000×g for 10 min. Thereafter, the supernatant was discarded, 5 mL of 50% ethyl alcohol was added to the residue which was then stirred and centrifuged at 2000×g for 10 min. After the resulting supernatant was decanted again, 5 mL of 2 M trifluoroacetic acid (TFA) was added to the residue which was then stirred and left in an oil bath at 120° C. for 40 min.

Next, the sample was cooled and neutralized with an addition of 5 mL of 2 N KOH. The sample was transferred to a 50 mL flask which was then filled with 50 mL of 0.1 M NaOAc (pH 4.5) buffer. The contents in the flask were stirred using a magnetic bar. Insoluble materials formed in the sample solution were removed by centrifugation. Herein, 50 μL of exo-1,3-β-glucanase (Megazyme, Wicklow, Ireland) and 50 μL of β-glucosidase (Megazyme, Wicklow, Ireland) were added to 100 μL of the sample solution, followed by a reaction at 40° C. for 60 min.

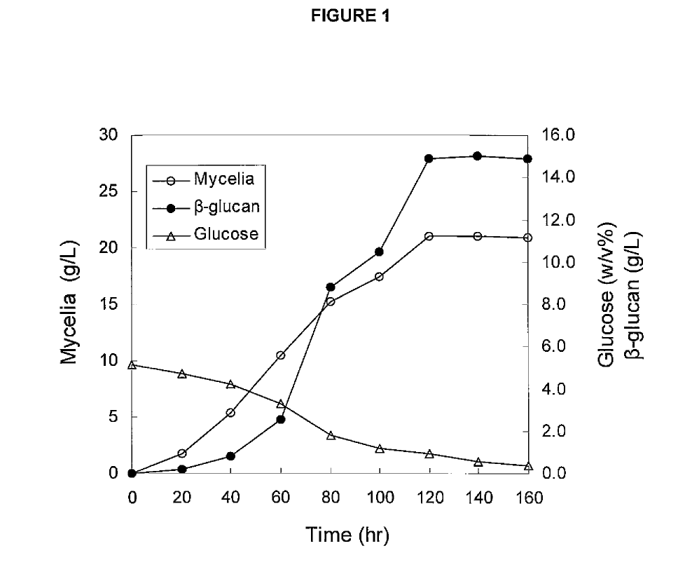

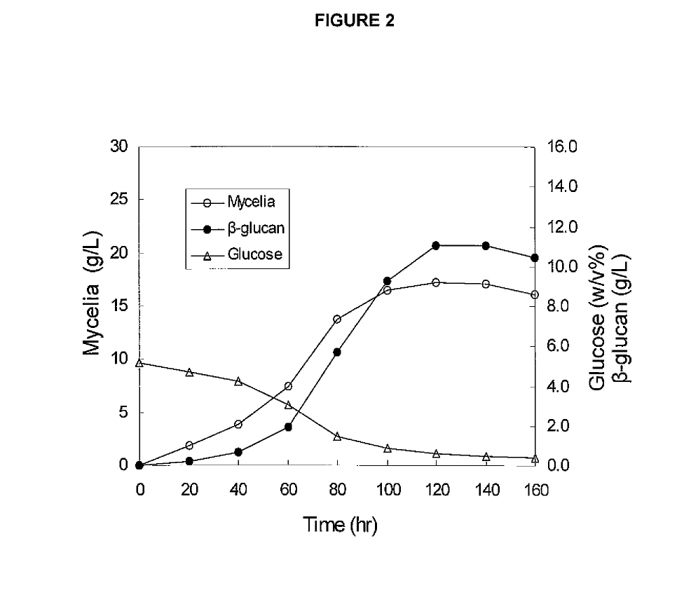

Then, glucose was assayed using a glucose assay kit (Glu CII, Wako, Tokyo, Japan) and calculated in terms of beta-glucan by a calculation formula. The thus-obtained results are shown in FIGS. 1 and 2, and Table 1.

FIG. 1 is a graph showing changes in time-dependent mycelial mass, polysaccharide production and glucose consumption, in order to examine the productivity (yield) of beta-glucan over time, upon the use of a synthetic adsorbent as in Example 1; and FIG. 2 is a graph showing changes in time-dependent mycelial mass, polysaccharide production and glucose consumption, in order to examine the productivity (yield) of beta-glucan over time, upon no use of a synthetic adsorbent as in Comparative Example 1.

Referring to FIGS. 1 and 2, it can be seen that 5-day-culture of Schizophyllum commune mycelia exhibits the highest productivity of beta-glucan. This result can be confirmed from Table 1. A weight-average molecular weight of the beta-glucan was separately determined by gel permeation chromatography (GPC).

TABLE 1

| | | | Weight- |
|---|---|---|---|
| | Mycelial | Beta-glucan | average molecular |
| Example No. | mass (g/L) | polysaccharide (g/L) | weight (MW) |
| Ex. 1 | 11.1 | 15.5 | 3,500,000 |
| Comp. Ex. 1 | 8.5 | 10.2 | 2,500,000 |

Referring to Table 1, Example 1 exhibited about a 51% improvement in the yield of beta-glucan polysaccharide (15.5 g/L), as compared to Comparative Example 1 (10.2 gL). This result arises from the fact that upon production of beta-glucan by liquid culture of Schizophyllum commune mycelia, addition of the synthetic adsorbent leads to uniform growth of the mycelia due to adhesiveness thereof, and the synthetic adsorbent adsorbs culture by-products, in conjunction with improved productivity of beta glucan.

Further, it can be seen that beta glucan of Example 1 obtained using the synthetic adsorbent exhibits a higher molecular weight, as compared to that of Comparative Example 1.

In addition, the analysis of the polysaccharide of Example 1 confirmed that it contains 99% beta glucan.

### EXPERIMENTAL EXAMPLE 2

Using beta glucans obtained in Example 2 and Comparative Example 2, an experiment was carried out in the same manner as in Experimental Example 1. The-thus obtained results are given in Table 2 below.

TABLE 2

| | | | Weight-average |
|---|---|---|---|
| | Mycelial mass | Beta-glucan | molecular |
| Example No. | (g/L) | polysaccharide (g/L) | weight (MW) |
| Ex. 2 | 12.4 | 15.9 | 3,000,000 |
| Comp. Ex. 2 | 9.2 | 10.0 | 2,500,000 |

Upon reviewing Table 2, similar to Experimental Example 2, it can be seen that beta glucan of Example 2 obtained using the synthetic adsorbent exhibits higher values in the yield and molecular weight, as compared to that of Comparative Example 2.

### EXPERIMENTAL EXAMPLE 3

In order to examine the productivity (yield) of beta-glucan upon the use of activated charcoal as in Comparative Example 3, an experiment was carried out in the same manner as in Experimental Example 1. The-thus obtained results are given in Table 3 below.

FIG. 3 is a graph showing changes in time-dependent mycelial mass, polysaccharide production and glucose consumption, in order to examine the productivity (yield) of beta-glucan over time, upon the use of activated charcoal as in Comparative Example 3. For comparison with Comparative Example 3, results of Examples 1 and 2 using the synthetic adsorbent are also given in Table 3 below.

TABLE 3

| | | Beta-glucan |
|---|---|---|
| Example No. | Mycelial mass (g/L) | polysaccharide (g/L) |
| Ex. 1 | 11.1 | 15.5 |
| Ex. 2 | 12.4 | 15.9 |
| Comp. Ex. 3 | 9.0 | 12.0 |

From the results shown in FIG. 3 and Table 3, it can be seen that the use of activated charcoal (Comparative Example 3) exhibited a significantly low yield of the beta-glucan polysaccharide, as compared to those of the beta-glucan polysaccharides of Examples 1 and 2.

### EXPERIMENTAL EXAMPLE 4

### Effects of Beta-Glucan on Synthesis of Filaggrin by Skin Cells

In order to examine production of a natural moisturizing factor (NMF) having an important effect on skin moisturization, synthesis of filaggrin, which is a precursor of the natural moisturizing factor, was tested on human skin cells.

First, skin cells were introduced into a culture flask and allowed to adhere to the bottom of the flask. Then, cells were cultured for 2 days by co-addition of 35-S-methionine upon treatment of a test material. After the cultivation was complete, the culture medium was removed and the cells were dissolved in phosphate buffered saline (PBS) containing 2% sodium dodecyl sulfate and 20 mM dithiothreitol (DTT).

After the skin cells were cultured, treatment of the cells with the test material was carried out for 2 days. After the test material treatment was complete, the medium was removed, and only the cells were taken and disrupted using an ultrasonic disintegrator to thereby obtain only a protein. Using the thus-obtained protein, an amount of synthesized filaggrin was determined by Western blotting analysis.

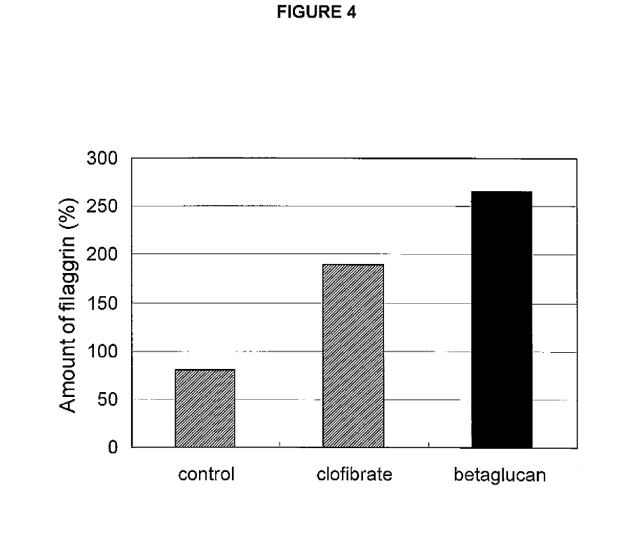

FIG. 4 is a graph showing an expression amount of filaggrin by Western blotting analysis. Clofibrate was used as a positive control, and beta-glucan was added to a concentration of 0.01% for the test.

As shown in FIG. 4, the synthesis of filaggrin significantly increased in the skin cells treated with beta-glucan. Therefore, an increase in the level of the natural moisturizing factor can be confirmed indirectly from the result showing increased synthesis of filaggrin which is the precursor of the natural moisturizing factor.

### EXPERIMENTAL EXAMPLE 5

### Effects of Beta-Glucan on Skin Moisture Loss Recovery

This experiment was carried out to evaluate effects of beta-glucan on recovery of a skin barrier function loss due to long-term skin damage.

A barrier function of dorsal skin of experimental animals was damaged by periodic application of acetone to the dorsal parts of young hairless mice (10 weeks old) for 3 consecutive days three times a day. Trans-epidermal water loss (TEWL) was measured in the experimental animals. For only the mice having skin with more than 40 g/m²/hr of TEWL, a vehicle (propylene glycol:water=5:5) and a sample containing 0.2% beta-glucan of Example 1 were applied at a dose of 200 μl per 5 cm² of a skin area for 3 consecutive days three times a day, and TEWL was measured periodically.

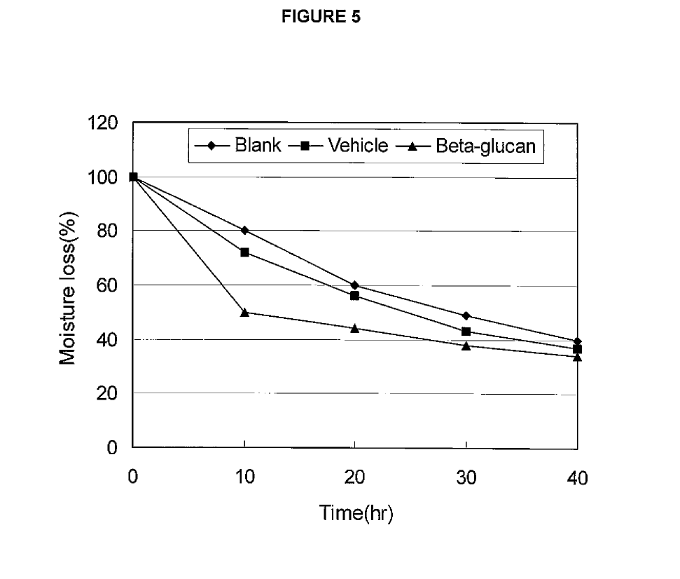

FIG. 5 is a graph showing moisture loss recovery over time by beta-glucan. Referring to FIG. 5, it can be seen that the beta-glucan treated group exhibits rapid recovery from the skin barrier damage to thereby return to normal skin, as compared to the vehicle group. Further, when the skin barrier damage occurred, recovery of the trans-epidermal water loss by beta-glucan after 10 hours was 50% or higher, thus showing remarkable recovery of the skin barrier function.

### EXPERIMENTAL EXAMPLE 6

### Effects of Beta-Glucan on Alleviation of Atopic Pruritus

This experiment was carried out to confirm effects of a beta-glucan-containing external application composition on alleviation of atopic dermatitis and pruritus.

First, a composition for external application comprising beta-glucan prepared in Example 2) was prepared according to the following composition formula given in Table 4 below. The thus-prepared external application composition was applied to intractable atopic patients. 50 patients, ranging from ages 2 to 20, were included in this experiment and were divided into 5 groups, each consisting of 10 patients. 1 min, 10 min and 1 hour after application of the composition, whether the subjects feel itchy was examined by the questionnairing. The number of the subjects complaining itching is given in Table 5 below. Itching was defined as a degree of a sensation felt on an area of skin that provokes the desire to rub or scratch the skin to obtain relief.

TABLE 4

(unit: wt %)

| Ingredients | Ex. 3 | Comp. Ex. 3 |
|---|---|---|
| Beta-glucan prepared in Example 2 | 0.1 | — |
| Wax | 3.0 | 3.0 |
| Polysorbate 60 | 1.5 | 1.5 |
| PEG-60 hydrogenated castor oil | 2.0 | 2.0 |
| Sorbitan sesquioleate | 0.5 | 0.5 |
| Liquid paraffin | 10.0 | 10.0 |
| Squalane | 5.0 | 5.0 |
| Caprylic/capric triglyceride | 5.0 | 5.0 |
| Glycerin | 5.0 | 5.0 |
| Butylene glycol | 3.0 | 3.0 |
| Propylene glycol | 3.0 | 3.0 |
| Triethanolamine | 0.2 | 0.2 |
| Preservative, Pigment, and perfume | q.s. | q.s. |
| Purified water | to 100 | to 100 |

TABLE 5
Effects of beta-glucan on amelioration of atopic pruritus

| | | | | | Composition |
|---|---|---|---|---|---|
| | | | | | of |
| | Composition of Ex. 3 | | | | Comp. Ex. 3 |
| Time | Group 1 | Group 2 | Group 3 | Group 4 | Group 5 |
| Itching after 1 min | 2 | 4 | 4 | 5 | 9 |
| Itching after 10 min | 3 | 5 | 6 | 6 | 10 |
| Itching after 1 hr | 4 | 5 | 7 | 6 | 10 |

Referring to Table 5, Group 1 with application of the beta-glucan-containing composition exhibited 80% puritus-relieving effects after 1 min, Groups 2 and 3 exhibited 60% pruritus-relieving effects and Group 4 exhibited 50% relieving effects of atopie puritus. On the other hand, Group 5 with application of the composition of Comparative Example 3 containing no beta-glucan exhibited substantial no pruritus-relieving effects.

Similar results were also observed 10 min and 1 hour after application of the composition. That is, as the administration of the composition of Example 3 exhibited excellent effects on alleviation of pruritus, it was determined that the composition, taking advantage of skin application and skin moisture loss recovery of beta-glucan, exerts excellent effects on alleviation of atopic pruritus.

### EXPERIMENTAL EXAMPLE 7

### Effects of Beta-Glucan on Amelioration of Pain and Itching Associated with Skin Damage Due to Viral Infections

This experiment was carried out to investigate effects of beta-glucan on amelioration of pain and itching associated with skin damage due to viral infections.

First, a composition for external application comprising beta-glucan (prepared in Example 2) was prepared according to the following composition formula given in Table 6 below. As Comparative Examples 4 to 6, the thus-prepared external application composition containing aspirin was applied to the affected parts of patients (n=24) who show viral infection conditions including vesicles, lesions or ulcers by viral infections of varicella-zoster virus (VZV) or herpes simplex virus (HSV), and suffer from pain and itching. Thereafter, the relief of pain and itching was evaluated.

As Examples 4 and 5 in accordance with the present invention, the external preparation containing beta-glucan for use in the treatment of such conditions was administered to the patients, and the relief of pain and itching was evaluated. Based on subjective feelings and symptoms of the patients, the pain and itching-relief degree was evaluated according to the following scale:

A: excellent, B: good, C: slight relief, D: no change, and E: getting worse

TABLE 6

| | (unit: wt %) | | | | |
|---|---|---|---|---|---|
| | Comp. | Comp. | Comp. | | |
| Ingredients | Ex. 4 | Ex. 5 | Ex. 6 | Ex. 4 | Ex. 5 |
| Beta-glucan of | — | — | — | 1.0 | 1.0 |
| Example 2 | | | | | |
| Aspirin | — | 2.0 | 1.0 | 1.0 | — |
| Wax | 3.0 | 3.0 | 3.0 | 3.0 | 3.0 |
| Polysorbate 60 | 1.5 | 1.5 | 1.5 | 1.5 | 1.5 |
| PEG-60 hydrogenated | 2.0 | 2.0 | 2.0 | 2.0 | 2.0 |
| castor oil | | | | | |
| Sorbitan sesquioleate | 0.5 | 0.5 | 0.5 | 0.5 | 0.5 |
| Liquid paraffin | 10.0 | 10.0 | 10.0 | 10.0 | 10.0 |
| Squalane | 5.0 | 5.0 | 5.0 | 5.0 | 5.0 |
| Caprylic/capric | 5.0 | 5.0 | 5.0 | 5.0 | 5.0 |
| triglyceride | | | | | |
| Glycerin | 5.0 | 5.0 | 5.0 | 5.0 | 5.0 |
| Butylene glycol | 3.0 | 3.0 | 3.0 | 3.0 | 3.0 |
| Propylene glycol | 3.0 | 3.0 | 3.0 | 3.0 | 3.0 |
| Triethanolamine | 0.2 | 0.2 | 0.2 | 0.2 | 0.2 |
| Preservative, Pigment, | q.s. | q.s. | q.s. | q.s. | q.s. |
| and perfume | | | | | |
| Purified water | to 100 | to 100 | to 100 | to 100 | to 100 |

TABLE 7
Amelioration of pain and itching in viral infection-induced
affected parts such as vesicles, lesions or ulcers by viral infections
of varicella-zoster virus or herpes simplex virus

| | Principal | Number of | Evaluation | | | | |
|---|---|---|---|---|---|---|---|
| Example No. | ingredients (%) | subjects | A | B | C | D | E |
| Comp. Ex. 4 | — | 2 | 0 | 0 | 0 | 1 | 1 |
| Comp. Ex. 5 | Aspirin 2 | 6 | 2 | 2 | 2 | 0 | 0 |
| Comp. Ex. 6 | Aspirin 1 | 4 | 1 | 2 | 0 | 1 | 0 |
| Ex. 4 | Aspirin 1 + beta- | 6 | 2 | 3 | 1 | 0 | 0 |
| | glucan 1 | | | | | | |
| Ex. 5 | Beta-glucan 1 | 6 | 1 | 3 | 2 | 0 | 0 |

Upon comparing with the composition of Comparative Example 4 with reference to Table 7, it can be seen that the beta-glucan-containing compositions of Examples 4 and 5 exhibited the inhibition degree of pain and itching comparable to or higher than that of the composition of Comparative Examples 5 and 6 containing aspirin alone, in affected parts of viral infection-induced damage such as vesicles, lesions or ulcers by viral infections of varicella-zoster virus or herpes simplex virus.

### EXPERIMENTAL EXAMPLE 8

### Effects of Beta-Glucan on Bacterial Inhibition in Mouse Model

Skin is directly vulnerable to or may suffer from single infection with various bacteria and about 90% of atopic patients suffer from secondary infections with bacteria such as Staphylococcus spp. Therefore, there is a need for bacterial inhibition and amelioration of infectious diseases. Prior to the test of skin itching due to bacterial infections, this experiment was carried out to test whether beta-glucan has direct synergistic effects on antibacterial activity and antibiotic activity in vivo, using a mouse model.

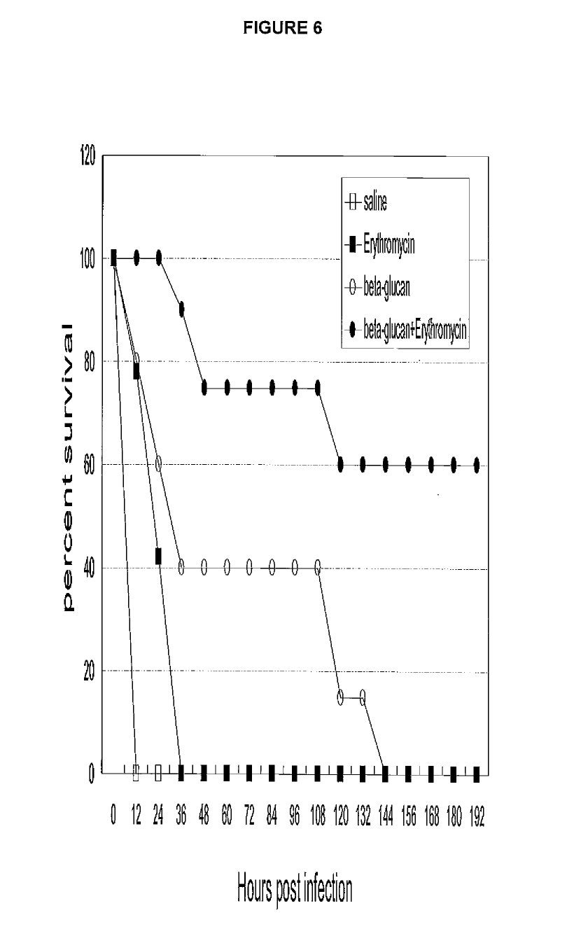

Erythromycin was used as an antibiotic and Staphylococcus aureus, one of staphylococci as a major cause of skin infections, was used as the test bacteria. Each experimental group was composed of 6 to 7 mice per group. Experiments were carried out as follows. The results thus obtained are shown in FIG. 6.

1) 3 days and 1 day prior to bacterial challenge, 1 mg/mouse of saline, antibiotic, beta-glucan, or a beta-glucan/antibiotic mixture was peritoneally injected into mice.

2) 30 min prior to bacterial administration, 18 mg/kg of the antibiotic was subcutaneously injected into the mice.

3) Then, 1×10⁹ of bacteria (S. aureus) were intravenously injected to induce septic shock.

4) Finally, a survival rate (%) of the animals was periodically confirmed every 12 hours.

FIG. 6 is a graph showing percent survival of mice after infection of animals with Staphylococcus aureus.

Referring to FIG. 6, the mice of the group with administration of the antibiotic or physiological saline alone were all killed within 48 hours following the bacterial infection, whereas the group with administration of beta-glucan alone exhibited a 40% survival rate at 48 hours after the bacterial infection, and the group with co-administration of beta-glucan with the antibiotic exhibited a 71% survival rate at 48 hours after the bacterial infection.

Further, one week after the bacterial infection, animals of the group with administration of physiological saline, antibiotic or beta-glucan alone were all dead, whereas the co-administration group of beta-glucan and antibiotic exhibited a 57% survival rate.

These results suggest that the beta-glucan in accordance with the present invention can function as an immunomodulator and therefore potentiate the antibacterial activity upon combined administration thereof with the antibiotic, and it is thus possible to more fundamentally relieve skin edema, pain and itching due to bacterial infections including S. aureus, or pain and itching of atopic diseases resulting from secondary bacterial infection.

### EXPERIMENTAL EXAMPLE 9

### Effects of Beta-Glucan on Amelioration of Pain and Itching Associated with Skin Damage Due to Bacterial Infections

First, a composition for external application comprising beta-glucan (prepared in Example 2) was prepared according to the following composition formula given in Table 8 below.

Among atopic patients, 25 subjects infected with skin pathogens Staphylococcus aureus or Streptococcus pyogenes were treated with the formulation containing beta-glucan or an antibiotic three times a day. Based on subjective feelings and symptoms of the patients after 3 days, the itching-relief degree was evaluated according to the following scale:

A: excellent, B: good, C: slight relief, D: no change, and E: getting worse.

The-thus obtained results are given in Table 9 below.

TABLE 8

| | (unit: wt %) | | | |
|---|---|---|---|---|
| | Comp. | Comp. | | |
| Ingredients | Ex. 7 | Ex. 8 | Ex. 6 | Ex. 7 |
| Beta-glucan of Example 2 | — | — | 1.0 | 1.0 |
| Antibiotic (Erythromycin) | — | 0.01 | 0.01 | — |
| Wax | 3.0 | 3.0 | 3.0 | 3.0 |
| Polysorbate 60 | 1.5 | 1.5 | 1.5 | 1.5 |
| PEG-60 hydrogenated castor oil | 2.0 | 2.0 | 2.0 | 2.0 |
| Sorbitan sesquioleate | 0.5 | 0.5 | 0.5 | 0.5 |
| Liquid paraffin | 10.0 | 10.0 | 10.0 | 10.0 |
| Squalane | 5.0 | 5.0 | 5.0 | 5.0 |
| Caprylic/capric triglyceride | 5.0 | 5.0 | 5.0 | 5.0 |
| Glycerin | 5.0 | 5.0 | 5.0 | 5.0 |
| Butylene glycol | 3.0 | 3.0 | 3.0 | 3.0 |
| Propylene glycol | 3.0 | 3.0 | 3.0 | 3.0 |
| Triethanolamine | 0.2 | 0.2 | 0.2 | 0.2 |
| Preservative, Pigment, and | q.s. | q.s. | q.s. | q.s. |
| perfume | | | | |
| Purified water | to 100 | to 100 | to 100 | to 100 |

TABLE 9
Relief of pain and itching in atopic patients with bacterial infections

| | | Number | | | | | |
|---|---|---|---|---|---|---|---|
| | Principal | of | Evaluation | | | | |
| Example No. | ingredients | subjects | A | B | C | D | E |
| Comp. Ex. 7 | — | 4 | 0 | 0 | 1 | 2 | 1 |
| Comp. Ex. 8 | Antibiotic | 7 | 0 | 2 | 2 | 2 | 1 |
| Ex. 6 | Antibiotic + beta- | 7 | 2 | 4 | 1 | 0 | 0 |
| | glucan | | | | | | |
| Ex. 7 | Beta-glucan | 7 | 2 | 3 | 2 | 0 | 0 |

Referring to Table 9, it can be seen that the composition of Comparative Example 7 containing no beta-glucan exhibits substantially no pain and itching-relieving effects on the affected parts of secondary infection patients.

On the other hand, the atopic patients with treatment of the compositions containing beta-glucan in accordance with the present invention (compositions of Examples 6 and 7) mostly exhibit the pain and itching-relief degree of A or B, thus representing a significant reduction of itching. These results show that compositions of Examples 6 and 7 are superior to that of Comparative Example 8 containing the antibiotic.

Hereinafter, the constitution of a composition for external application containing Schizophyllum commune-derived beta-glucan prepared in Example 1 will be described in more detail with reference to the following Formulation Examples 1 to 11. However, it should be understood that the composition of the present invention is not limited to these Formulation Examples.

### FORMULATION EXAMPLE 1

### Emollient Lotion (Skin Lotion)

According to the following composition formula given in Table 10 below, a skin lotion containing beta-glucan was prepared by a conventional method.

TABLE 10

| Ingredients | Unit (wt %) |
|---|---|
| Beta-glucan | 0.1 |
| Glycerin | 3.0 |
| Propylene glycol | 3.0 |
| Carboxyvinyl polymer | 0.1 |
| PEG-12 nonylphenyl ether | 0.2 |
| Polysorbate 80 | 0.5 |
| Ethanol | 10.0 |
| Triethanolamine | 0.1 |
| Preservative, Pigment, and perfume | q.s. |
| Purified water | to 100 |

### FORMULATION EXAMPLE 2

### Nourishing Lotion (Milk Lotion)

According to the following composition formula given in Table 11 below, a milk lotion containing beta-glucan was prepared by a conventional method.

TABLE 11

| Ingredients | Unit (wt %) |
|---|---|
| Beta-glucan | 0.2 |
| Squalane | 5.0 |
| Wax | 2.0 |
| Polysorbate 60 | 1.5 |
| Sorbitan sesquioleate | 1.5 |
| Liquid paraffin | 1.5 |
| Caprylic/capric triglyceride | 5.0 |
| Glycerin | 5.0 |
| Butylene glycol | 3.0 |
| Propylene glycol | 3.0 |
| Carboxyvinyl polymer | 0.1 |
| Triethanolamine | 0.2 |
| Preservative, Pigment, and perfume | q.s. |
| Purified water | to 100 |

### FORMULATION EXAMPLE 3

### Nourishing Cream

According to the following composition formula given in Table 12 below, a nourishing cream containing beta-glucan was prepared by a conventional method.

TABLE 12

| Ingredients | (unit: wt %) |
|---|---|
| Beta-glucan | 0.5 |
| Wax | 10.0 |
| Polysorbate 60 | 1.5 |
| PEG-60 hydrogenated castor oil | 1.0 |
| Sorbitan sesquioleate | 0.5 |
| Liquid paraffin | 10.0 |
| Squalane | 3.0 |
| Caprylic/capric triglyceride | 3.0 |
| Glycerin | 5.0 |
| Butylene glycol | 3.0 |
| Propylene glycol | 3.0 |
| Triethanolamine | 0.2 |
| Preservative, Pigment, and perfume | q.s. |
| Purified water | to 100 |

### FORMULATION EXAMPLE 4

### Massage Cream

According to the following composition formula given in Table 13 below, a massage cream containing beta-glucan was prepared by a conventional method.

TABLE 13

| Ingredients | (unit: wt %) |
|---|---|
| Beta-glucan | 1.0 |
| Wax | 10.0 |
| Polysorbate 60 | 1.0 |
| PEG-60 hydrogenated castor oil | 2.0 |
| Sorbitan sesquioleate | 0.8 |
| Liquid paraffin | 30.0 |
| Squalane | 5.0 |
| Caprylic/capric triglyceride | 3.0 |
| Glycerin | 5.0 |
| Butylene glycol | 3.0 |
| Propylene glycol | 3.0 |
| Triethanolamine | 0.2 |
| Preservative, Pigment, and perfume | q.s. |
| Purified water | to 100 |

### FORMULATION EXAMPLE 5

### Pack

According to the following composition formula given in Table 14 below, a pack containing beta-glucan was prepared by a conventional method.

TABLE 14

| Ingredients | (unit: wt %) |
|---|---|
| Beta-glucan | 0.5 |
| Polyvinyl alcohol | 10.0 |
| Sodium carboxymethyl cellulose | 0.2 |
| Glycerin | 5.0 |
| Allantoin | 0.1 |
| Ethanol | 5.0 |
| PEG-12 nonylphenyl ether | 0.3 |
| Polysorbate 60 | 0.3 |
| Preservative, Pigment, and perfume | q.s. |
| Purified water | to 100 |

### FORMULATION EXAMPLE 6

### Gel

According to the following composition formula given in Table 15 below, a gel containing beta-glucan was prepared by a conventional method.

TABLE 15

| Ingredients | Unit (wt %) |
|---|---|
| Beta-glucan | 1.0 |
| Sodium ethylenediamine | 0.02 |
| acetate | |
| Glycerin | 5.0 |
| Carboxyvinyl polymer | 0.6 |
| Ethanol | 5.0 |
| PEG-60 hydrogenated castor | 0.5 |
| oil | |
| Triethanolamine | 0.2 |
| Preservative, Pigment, and | q.s. |
| perfume | |
| Purified water | to 100 |

### FORMULATION EXAMPLE 7

### Ointment

According to the following composition formula given in Table 16 below, an ointment containing beta-glucan was prepared by a conventional method.

TABLE 16

| Ingredients | (unit: wt %) |
|---|---|
| Beta-glucan | 2.0 |
| Wax | 10 |
| Polysorbate 60 | 5.0 |
| PEG-60 hydrogenated castor oil | 3.0 |
| Sorbitan sesquioleate | 0.5 |
| Vaseline | 6.0 |
| Liquid paraffin | 10.0 |
| Squalane | 3.0 |
| Shea butter | 5.0 |
| Caprylic/capric triglyceride | 5.0 |
| Glycerin | 10.0 |
| Propylene glycol | 5.0 |
| Triethanolamine | 0.2 |
| Preservative, Pigment, and perfume | q.s. |
| Purified water | to 100 |

### FORMULATION EXAMPLE 8

### Topical Preparation (Gel Ointment)

According to the following composition formula given in Table 17 below, a topical preparation containing beta-glucan was prepared by a conventional method.

TABLE 17

| Ingredients | (unit: wt %) |
|---|---|
| Beta-glucan | 5.0 |
| Polyacrylic acid (Carbopol 940) | 1.0 |
| Isopropanol | 5.0 |
| Hexylene glycol | 25.0 |
| Triethanolamine | 1.5 |
| Deionized water | to 100 |

### FORMULATION EXAMPLE 9

### Topical Preparation (Patch)

According to the following composition formula given in Table 18 below, a topical preparation containing beta-glucan was prepared by a conventional method.

TABLE 18

| Ingredients | (unit: wt %) |
|---|---|
| Beta-glucan | 1.0 |
| Hexylene glycol | 15.0 |
| Diethyl amine | 0.5 |
| Polyacrylic acid (Carbopol 934P) | 1.0 |
| Sodium sulfite | 0.1 |
| Polyoxyethylene lauryl ether | 1.0 |
| Polyhydroxyethylene cetyl stearyl ether | 1.0 |
| Paraffin oil | 3.5 |
| Caprylic ester/capric ester | 3.0 |
| Polyethylene glycol 400 | 3.0 |
| Deionized water | to 100 |

### FORMULATION EXAMPLE 10

### Shampoo-Type Cleanser

According to the following composition formula given in Table 19 below, a shampoo-type cleanser containing beta-glucan was prepared by a conventional method.

TABLE 19

| Ingredients | (unit: wt %) |
|---|---|
| Beta-glucan | 1.0 |
| Lauryl diethanol amide | 6.0 |
| Propylene glycol | 1.0 |
| Citric acid | 0.1 |
| Sodium lauryl sulfate solution | 15.0 |
| Sodium polyoxyethylene lauryl ether sulfate | 30.0 |
| Preservative, Pigment, and perfume | q.s. |
| Deionized water | to 100 |

### FORMULATION EXAMPLE 11

### Skin Cleanser

According to the following composition formula given in Table 20 below, a skin cleanser containing beta-glucan was prepared by a conventional method.

TABLE 20

| Ingredients | (unit: wt %) |
|---|---|
| Beta-glucan | 2.0 |
| Sodium lauryl sulfate (30%) | 16.0 |
| Sodium lauryl ether sulfate (30%) | 11.0 |
| Coconut diethanolamide | 4.0 |
| Myristic acid | 2.0 |
| Lauric acid | 2.0 |
| Preservative, Pigment, and perfume | q.s. |
| Glycerin | 3.0 |
| Triethanolamine | 2.0 |
| Deionized water | to 100 |

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition for external application having relieving effects on skin dryness conditions, pruritus and pain, and comprising Schizophyllum commune-derived beta-glucan, wherein the content of the Schizophyllum commune-derived beta-glucan is in the range of 0.01 to 20% by weight, based on the total weight of the composition.

2. The composition according to claim 1, wherein the beta-glucan is a beta-1,6-branched-beta-1,3-glucan having a beta-1,6-residue for every three beta-1,3 main chains.

3. The composition according to claim 1, wherein the beta-glucan has a weight-average molecular weight of 3,000,000 to 4,000,000.

4. The composition according to claim 1, wherein the pruritus and pain are caused by atopic pruritus or by a secondary bacterial infection.

5. The composition according to claim 4, wherein the secondary bacterial infection is an infection by one selected from the group consisting of Staphylococcus aureus, Streptococcus pyogenes, and any combination thereof.

6. The composition according to claim 1, wherein the pruritus and pain are caused by a bacterial infection.

7. The composition according to claim 6, wherein the bacterial infection is caused by Staphylococcus aureus.

8. The composition according to claim 1, wherein the pruritus and pain are caused by viral infection.

9. The composition according to claim 8, wherein the viral infection is caused by chicken pox virus, herpes zoster virus, or herpes simplex virus.

10. The composition according to claim 1, wherein the pruritus and pain include senile pruritus, and pruritus and pain in vesicles, lesions or ulcers of skin mucosa by bacterial or viral infections.

11. The composition according to claim 1, wherein the composition for external application is a cosmetic composition having a formulation of an emollient lotion (skin lotion), a nourishing lotion (milk lotion), a nourishing cream, a massage cream, a pack or a gel.

12. The composition according to claim 1, wherein the composition for external application is a composition having a formulation of a body lotion, an ointment, a gel, a cream, a patch, a shampoo-type cleanser, a skin cleanser or a spray.

13. A method for producing Schizophyllum commune-derived beta-glucan, comprising culturing mycelia of Schizophyllum commune in a liquid culture medium with an addition of a synthetic adsorbent.

14. The method according to claim 13, wherein the synthetic adsorbent is one selected from the group consisting of a non-ionic styrene-divinyl benzene polymer, a phenol-formalin resin, an acrylate resin, a methacrylate resin and any combination thereof.

15. The method according to claim 13, wherein the synthetic adsorbent has a particle diameter of 0.1 to 1 mm.

16. The method according to claim 13, wherein the synthetic adsorbent has a particle diameter of 0.3 to 0.6 mm.

17. The method according to claim 13, wherein the synthetic adsorbent is one selected from the group consisting of Amberlite XAD-1, Amberlite XAD-2, Amberlite XAD-4, Amberlite XAD-7, Amberlite XAD-8, Amberlite XAD-11, Amberlite XAD-12, Diaion HP-10, Diaion HP-20, Diaion HP-30, Diaion HP-40, Diaion HP-50, Imac syn-42, Imac syn-44, Imac syn-46 and any combination thereof.

18. The method according to claim 13, wherein the Schizophyllum commune-derived beta-glucan is produced by a process comprising:
1) inoculating a seed obtained by culturing the mycelia of Schizophyllum commune in an amount of 1 to 10% (v/v) into a liquid medium;
2) culturing the mycelial cells for 4 to 8 days with an addition of a sterile synthetic adsorbent in an amount of 1 to 10%, based on the total weight of the medium;
3) centrifuging the thus-obtained culture solution to remove the mycelia and the synthetic adsorbent; and
4) recovering, separating and purifying beta-glucan from the resulting beta-glucan solution.

19. The method according to claim 18, wherein the medium contains 1 to 20% glucose, 0.1 to 5% yeast extract, 0.1 to 5% malt extract, 0.1 to 1% ammonium sulfate ((NH₄)₂SO₄), 0.1 to 1% potassium dihydrogen phosphate (KH₂PO₄), and 0.01 to 0.5% magnesium sulfate (MgSO₄.7H₂O).

20. The method according to claim 18, wherein the culturing process is carried out at a temperature of 20 to 35° C., an agitation rate of 100 to 400 rpm, and an aeration rate of 0.5 to 2 vvm.
